(19) United States
(12) Patent Application Publication
(10) Pub. No.: US 2013/0211159 A1
(43) Pub. Date: Aug. 15, 2013

(54) METHOD FOR DEGASSING A STORAGE TANK FOR LIQUID PRODUCTS CONTAINING VOLATILE ORGANIC COMPOUNDS, IN PARTICULAR PETROLEUM PRODUCTS

(71) Applicant: Ivo Saverio Rancich, Cogoleto (IT)

(72) Inventor: Ivo Saverio Rancich, Cogoleto (IT)

(73) Assignee: IDRABEL ITALIA S.R.L., Savona (IT)

(21) Appl. No.: 13/698,611

(22) PCT Filed: May 18, 2010
(86) PCT No.: PCT/IT2010/000219

§ 371 Date: Mar. 28, 2013

(51) Int. Cl. C07C 7/20

(52) U.S. Cl. CPC C07C 7/20
USPC 585/4; 585/2; 585/3; 422/243

(57) ABSTRACT

Method for degassing a storage tank for liquid products containing volatile organic compounds or VOC, in particular petroleum products, comprising a step a) of introducing an aqueous mixture comprising substances able to interact chemically and/or physically with the VOC in the gaseous state inside the tank, which is kept closed, so as to encourage them to pass from the gaseous phase to the liquid phase, and to remain in the liquid phase. Such introduction step being protracted so as to reduce the concentration of the VOC to a predetermined value functional to having an acceptable explosive limit.

### FIELD OF APPLICATION

The present invention relates to a method for degassing a storage tank for liquid products containing volatile organic compounds.

Advantageously, the method according to the invention can be used, in particular, for degassing storage tanks of petroleum products, crude or refined, such as crude oil, petrol or gasoil, and petrochemical products.

### STATE OF THE ART

The problem with degassing storage tanks arises prior to opening the same for cleaning, inspection and servicing operations. Tanks used for the storage of petroleum and petrochemical products, which contain products of a certain volatility such as to pose risks of explosion, are all prone to such problem.

The steps mentioned of degassing and opening are critical moments and a source of constant anxiety due to the dangerousness of the operation and related risks.

One degassing procedure often used envisages flushing with air. To such end extractors, or ejectors placed on the mobile roof of the tank are used.

Unfortunately, in the petrochemical sector, the number of accidents resulting in serious events occurring precisely following degassing and opening of the tanks is high. In these steps there is always a risk of uncontrolled emission and diffusion of inflammable and explosive gases within a certain area. The control and working procedures adopted do not always succeed in entirely mitigating the risks, in part because the diffusion of the gas transported by the wind may reach adjacent areas where the preventive expedients adopted in the workplace have not been applied and a triggering agent of any type may cause serious accidents.

One alternative procedure, used to considerably reduce the risks emphasised above, envisages inertisation of the atmosphere inside the tank before degassing, for example by introducing nitrogen or argon into the tank through openings on the roof.

As well as being expensive this operation may considerably lengthen degassing times. In fact, inertisation requires the prior sealing of all the gaskets of the tank, including the legs supporting the mobile roof. These operations of preparing the tank are in themselves delicate and lengthy considering in addition that personnel have to operate in a confined space (the roof of the tank). The vaporisation of the nitrogen, the dispersion of the nitrogen inside of the tank and the periodic test of control to be carried out during the procedure to check the level of inertisation achieved, all add to the length of the degassing procedure.

The degassing of the tanks, both in the case of flushing with air and in the case of inertisation, present, in addition, problems of an environmental nature connected with the release into the atmosphere of volatile organic substances.

### PRESENTATION OF THE INVENTION

The purpose of the present invention is to provide for a method for degassing a storage tank for liquid products containing volatile organic compounds which makes it possible to considerably reduce the risks of explosion without necessarily having to inertise the tank.

A further purpose of the present invention is to provide for a method for degassing a storage tank for liquid products containing volatile organic compounds, which makes it possible to considerably reduce degassing times, whilst operating safely.

A further purpose of the present invention is to provide for a method for degassing a storage tank for liquid products containing volatile organic compounds, which makes it possible to eliminate or considerably reduce the release of VOC into the atmosphere.

A further purpose of the present invention is to provide for a method for degassing a storage tank for liquid products containing volatile organic compounds, which does not require opening of the tank towards the atmosphere before completion of the degassing.

A further purpose of the present invention is to provide for a method for degassing a storage tank for liquid products containing volatile organic compounds, which can be conducted jointly with treatment of the bottom sludges of the tank so as to save time.

A further purpose of the present invention is to provide for a method for degassing a storage tank for liquid products containing volatile organic compounds, which is operatively simple and safe and can be procedurised.

### BRIEF DESCRIPTION OF THE DRAWINGS

The technical characteristics of the invention, according to the aforesaid purposes are evident from the contents of the claims made and will be made clearer in the detailed description below made with reference to the attached drawings representing by way of a non-limiting example one or more embodiments wherein:

FIG. 1 shows a flow diagram of a preferred form of application of the method according to the present invention;

FIG. 2 shows a diagram of a plant for implementing the method according to the present invention in the form of application shown in FIG. 1.

### DETAILED DESCRIPTION

The present invention relates to a method for degassing a storage tank for liquid products containing volatile organic compounds.

Advantageously, the method according to the invention can be used, in particular, to perform degassing of storage tanks of petroleum products, crude or refined, such as crude oil, petrol or gasoil, and petrochemical products.

According to a general embodiment of the invention, the method for degassing a storage tank for liquid products containing volatile organic compounds or VOC comprises a step a) of introducing inside a tank T, which is kept closed, an aqueous mixture comprising substances able to interact chemically and/or physically with the VOC in the gaseous state so as to encourage them both to pass from the gaseous phase to the liquid phase, and to remain in the liquid phase. Such introduction step is protracted until the concentration of the VOC is reduced to below a predetermined value functional to having an acceptable explosive limit for the purposes of being able to open the tank in safety for internal inspections.

A person skilled in the art knows how to assess the explosive limits of a gaseous mixture of compounds, with reference for example, to the LEL (Lower Explosive Limit) and UEL (Upper Explosive Limit).

By way of example, for storage tanks of crude oil according to some procedures, for the purposes of safety, an explosive limit of 10% may be considered acceptable.

The method according to the invention therefore envisages abatement of the volatile organic substances (hereinafter for simplicity's sake, called VOC, Volatile Organic Compounds) from the gaseous phase to the liquid phase directly inside the tank which is kept closed and therefore in a confined environment, until the concentrations of the VOC have been reduced to such levels as to entail risks of explosion considered acceptable.

By specifying that during step a) of introducing the aqueous mixture the tank is kept closed it means that the inner volume of the tank can be considered as a confined space and thereby not communicating in a free or uncontrolled manner with the outside environment. Therefore it can be envisaged that the inner volume of the tank is placed, temporarily or otherwise, in fluidic communication with another confined space outside the tank.

The step a) of introducing the aqueous mixture may be protracted for a variable period of time depending on the characteristics of the tank being degassed and of the products stored in it (and therefore depending, for example, on the size of the tank, the type of VOC to be abated, the product contained in the tank, the temperature of the residual concentrations of VOC in gaseous phase to be achieved, of the explosive limits considered acceptable), as well as depending on the reagent substances used present in the aqueous mixture, of their concentrations in the said mixture and on the ways of introduction of the aqueous mixture inside the tank.

As will be explained further below, first of all this makes it possible to conduct the degassing and/or opening of the tank in conditions of safety. Moreover, abatement of the VOC in liquid phase makes it possible to considerably limit the emissions of VOC in the atmosphere, with evident benefits from an environmental point of view.

The mixing of the aqueous mixture with any liquid residues and/or semi-solid residues of the product stored in the tank makes it possible in addition to prevent further vaporisation of the lightweight components present (VOC) in the liquid and/or semi-solid residues.

As will be explained further below, abatement of the VOC inside the tank makes it possible to considerably reduce degassing times (i.e. extraction of the gas) compared to the traditional methods which envisage the extraction of the gas by circulation of the air and/or inertisation (for example with N₂).

Preferably, the aforesaid aqueous mixture comprises one or more surfactant substances.

Operatively, such substances have the function of encouraging both the passage of the VOC from the gaseous phase to the liquid phase, and keeping them in the liquid phase, preventing volatility.

Such one or more surfactant substances may be chosen preferably in the group comprising alkylphenol ethoxylate, ethoxylate alcohol, sodium metasilicate, sodium dioctylsulphosuccinate, polypropylene glycol, alkyl-diphenyl-ether disulphonate, polyoxyethylene stirilfenil ether sulphate, polyoxymethylene alkyl ethers, polyoxyethylene alkyl amines, polyoxyethylene sorbitan laurates, polyoxyethylene sorbitan stearates, polyoxyethylene sorbitan oleates, polyoxyethylene sorbitan trioleates, polyoxyethylene stirilfenil ethers, carboxy betaine, benzyl ammonium salts and/or imidazolin laurate

Advantageously, the aqueous mixture may comprise one or more biosurfactants, in the presence or not of the aforesaid surfactants.

Such one or more biosurfactants may be chosen preferably from the group comprising glycolipids, lipopeptides, lipopolysaccharides, phosopholipids, fatty acids/neutral lipids and/or proteins.

Preferably, the aforesaid surfactants and/or biosurfactants (mixed together or used separately) are present in the aqueous mixture in a percentage in weight of the total of 0.002% to 0.006%.

Preferably, separately or jointly with the surfactants and/or biosurfactants, the aforesaid aqueous mixture may comprise one or more chelating substances.

Operatively, such one or more chelating substances have the function of encouraging, by chemically interacting with the VOC, the capture of the same by the aqueous mixture and therefore their absorption in the said mixture. This way both the VOC are encouraged both to pass from the gaseous phase to the liquid phase, and to remain in the liquid phase.

Such one or more chelating substances may be chosen preferably from the group comprising

ethylenediamine tetraacetic acid (EDTA) and derivatives, acetyl acetate, polyamines, oxalates, carbonates, crown ethers, 2,2′-bipiridin, glycinate, nitrylotriacetate, dimercaptopropanol, penicillamine, deferoxamine mexylate, dimercaptosuccinic acid, trientine and/or Chlorella vulgaris green unicellular water microalga

Preferably, such one or more chelating substances are present in the aqueous mixture with a percentage in weight of the total of 0.001% to 0.004%.

According to a preferred form of application of the invention, the aforesaid aqueous mixture comprises both surfactant and/or biosurfactants, and chelating substances. In fact a synergic effect has been observed deriving from the action of the two types of agent as regards absorbing the VOC and keeping them in the liquid phase.

Preferably, in the aforesaid aqueous mixture the chelating substances and the surfactant and/or biosurfactants are present in the aqueous mixture with a percentage in weight of the total of 0.003% to 0.01%.

According to a preferred form of application of the invention, the introduction of the aforesaid aqueous mixture inside the tank T is performed by means of one or more devices for generating pressurised jets inside the tank itself.

Advantageously, the aforesaid introduction step a) involves spraying the aqueous mixture inside the tank by means of the aforesaid pressurised jets.

Preferably, the spraying of the aqueous mixture by means of the aforesaid pressurised jets is performed to affect the entire volume of the tank, so as to permit the interaction of the mixture with the entire internal gaseous atmosphere.

Operatively, the entire inner volume of the tank need not necessarily be sprayed with the aqueous mixture in the same moment, but advantageously may be covered over a period of time, for example with cyclical covering of one or more volumetric portions.

Advantageously, the spraying of the aqueous mixture inside the tank may lead to at least a partial nebulisation of the aqueous mixture itself. The positive effect of the nebulisation lies in the greater persistence in the air of the aqueous mixture, finely separated into micro-drops, with an increase of the useful exchange surface for capturing the VOC.

The nebulisation may derive, for example, from the impact of the jets on the inner surfaces of the tank and/or as a secondary effect related to the fluid-dynamics of the jets themselves. Advantageously, it is possible to intervene on the fluid-dynamic characteristics of the jets (by acting on the pump and/or regulating the aperture of the nozzle generating the jet) to increase or reduce the nebulisation of the aqueous mixture.

According to a particularly preferred form of application of the present invention, the introduction step a) envisages the washing of the inner surfaces of the tank with the aqueous mixture by means of the aforesaid pressurised jets.

Operatively, such washing is for the purpose of cleaning the residues of the stored product from the inner surfaces of the tank, and in particular the roof and side walls. If not sufficiently treated in fact such surfaces are areas from which VOCs are released.

Preferably, washing is performed at the beginning of the introduction step a) of the aqueous mixture. The initial and preventive washing is performed so as to limit—during abatement treatment—the sources of emission of VOC to the residues at the bottom only, which remains in any case under the continuous action of the aqueous mixture gathering on the bottom of the tank.

As explained above, the introduction of the aforesaid aqueous mixture inside the tank T is performed by means of one or more devices for generating pressurised jets inside the tank itself.

Preferably, the method according to the invention comprises an step b) of inserting such one or more devices inside the tank by means of watertight chambers. Advantageously, the airtight chambers are connected to the blind flanges of the manholes of the tank and communicate with the inside of the tank itself through holes made on the flanges using safe cutting techniques, such as cold cutting. The connection to the flanges of the manholes is made using attachment bolts, without any welding.

One method for inserting devices generating pressurised jets through watertight chambers at the manholes is described, for example, in the International Patent application WO2008/114290A1 in the name of the same Applicant. The contents of such International Application are incorporated herein for reference.

Advantageously, as devices for generating pressurised jets, guns can be used with a nozzle which can be directed transversely and upwards to vary the range and direction of the jets.

The use of this type of orientable device is particularly advantageous for the purposes of the present invention given that it makes it possible to substantially treat the entire volume of the tank with the jets generated by it

In particular, each gun comprises a support structure which can be inserted through an aperture made in a manhole inside a watertight chamber connected to the blind flange of the manhole. The watertight chamber is provided with valve means to enable connection of the internal supply ducts of the nozzle with external pumping and collection means of the fluids to be introduced (in particular the aforesaid aqueous mixture) by means of the aforesaid guns.

In particular, the recycling of the aqueous mixture collected in the bottom of the tank to the guns may be envisaged, for example by connection to the drainage pipes in the bottom of the tank.

Advantageously, the method according to the invention comprises a step c) of monitoring at least the concentration of the VOC and/or the explosive limits inside the tank. Monitoring may also be extended to other potentially hazardous gaseous components, subject to abatement or not, for example sulphidric acid (H₂S).

Preferably, monitoring is conducted on at least one flow of gas extracted from the tank, using for example extraction lines associated to the aforesaid devices for generating pressurised jets.

As will be described further below, monitoring may be conducted, in addition to or alternatively, by taking samples of gas coming from the tank and collected in external confined spaces communicating with the tank, such as washing columns outside the tank.

Operatively, the monitoring step c) may be conducted prior, during or subsequent to completion of the introduction step a) of the aqueous mixture. Preferably, monitoring is repeated at predetermined intervals of time so as to establish over time the efficacy of the treatment and level of advancement of the VOC abatement.

According to a particularly preferred form of application of the invention, jointly or separately from the above, the method according to the invention comprises an step d) of extracting the residual gases present in the tank at the end of the aforesaid introduction step a).

Preferably, the extraction of the residual gases present in the tank is conducted using the same devices for generating pressurised jets used for the introduction step a). Such devices will be fluidically connected to aspiration means, such as fans or aspirators.

Operatively, the nozzle and the associated supply duct of the individual device generating the jets are therefore used as extraction ducts of the gas. Thanks to the aforesaid valve means which such devices may be fitted with, it is possible moreover to contemporaneously connect them both to extraction lines of the gas, and to supply lines of the fluids to be introduced inside the tank.

The step d) of extracting the gas is followed by a step f) of releasing the residual gases extracted from the tank into the atmosphere.

More specifically, at the end of the introduction step a) of the aqueous mixture the residual gases present in the tank may be extracted and released directly into the atmosphere with the opening of the tank or, according to a particularly preferred application of the present invention, they may be extracted and treated further, outside the tank, in one or more specially provided confined spaces before being released into the atmosphere.

Preferably, the method according to the invention may comprises an step e) of abating the VOC present in the residual gases extracted from the tank, subsequent to the extraction step d) of the residual gases and prior to the release step f).

The abatement step e) may be conducted as an extension of the introduction step a) of the aqueous mixture for the purpose of refining the abatement of the VOC in relation to the safety requisites and/or as a separate step aimed to improve the parameters of the residual gases coming out of the tank in relation to environmental problems, in the case in which the safety requirements for the risk of explosion have been already satisfied, but not the environmental requirements in relation to the concentrations of VOC.

More specifically, the abatement of the VOC may be conducted, for example:
by combustion with conveying of the gas to a flare;
by condensation using for example, cryogenic systems;
by absorption in the liquid phase using one or more washing columns; and/or
by absorption with activated carbon.

Combinations of the abatement methods listed above may be envisaged.

One preferred form of application of the invention envisages abatement by absorption in the liquid phase.

Preferably, such abatement is conducted using the aforesaid aqueous mixture as absorption liquid. To such purpose preferably fresh aqueous mixture is used. The aqueous mixture already introduced inside the tank and intercepted by means of the drainage ducts provided on the bottom of the tank may however be re-utilised, entirely or in part, supplemented or not with fresh aqueous solution.

Advantageously, alternatively or in addition to the monitoring conducted in relation to the introduction step a), the monitoring step c) may be conducted prior, during or at the end of the aforesaid abatement step e) of the VOC in the residual gases extracted, and preferably repeated at predefined intervals of time.

In particular, monitoring may be conducted by means of a variable speed/flow pneumatic extractor and by means of a sampling probe connected to multi-gas detection means for H₂S, LEL (Lower Explosive Limit), O² and to a photo-ionisation analyser for VOC. In the specific case of abatement by absorption in liquid phase using one or more washing columns, the probe may be positioned at the top of a washing column.

Advantageously, the monitoring of the gas in the tank may be conducted only in relation to the introduction step a), only in relation to the abatement step e) of the VOC, or in relation to both steps.

In the third case just pointed out, the monitoring conducted in relation to the abatement step e) may be conducted both as a further safety control of the gas in relation to the risk connected with the explosion of the gaseous flow, and as a control of the contents of pollutants/VOC in relation to risks of environmental pollution before emission into the atmosphere.

Preferably, the release into the atmosphere of the residual gases occurs in function of the concentrations measured in the aforesaid monitoring step c).

According to a preferred application of the present invention, the monitoring step c) is conducted throughout the entire degassing procedure, continuously or at predefined time intervals.

Advantageously, the measurement of the concentration of the VOC and/or of the explosive limits of the gases present inside the tank may be conducted using the measurement means which the devices for the VOC abatement step e) are provided for. Specifically, the measurement means associated with the washing column described above may be used. In this case, from an operative point of view, during the introduction step a) as mentioned above, a limited flow of gas is extracted from the tank and sent to the washing column for measurement. The washing column is not activated. Once the measurement has been made and the composition of the gas inside the tank has therefore been assessed, the washing column may be temporarily activated to abate the VOC present in the sample of gas extracted and then be released into the atmosphere once the desired safety and environmental protection conditions have been achieved.

Advantageously, the introduction step a) of the aqueous mixture may be preceded by a step g) of treating the bottom sludges present in the tank.

Tanks for storing liquid state products must be regularly subject to inspection, cleaning and maintenance operations for preventing the accumulation of sludge on the bottom, consisting of the heavier solid phases and/or of the liquid phases present in the stored liquids. During the storage, in fact, liquids are kept under ideal rest conditions for favouring settling and precipitation of the heavier components.

This problem is especially strong in tanks for crude oil storage. In fact, crude oil exhibits considerable amounts of solid materials in suspension (sands, rocky debris, metal salts and oxides, etc.) from the extraction sites. Moreover, the heavier hydrocarbon fractions of crude oil (naphthenic compounds and asphalts) naturally tend to aggregate and precipitate.

Advantageously, the method according to the invention may comprises the aforesaid treatment step g) of the bottom sludges comprising in turn the following sub-steps:

g1) breaking up the bottom sludge the bottom sludges by means of one or more pressurised jets;

g2) fluidising the bottom sludges by mixing by the action of said jets;

g3) dispersing biosurfactants able of emulsifying heavier hydrocarbon fractions, reducing the viscosity thereof and the tendency to precipitate;

g4) settling the contents of said tank interrupting the mixing thereof, so as to allow the precipitation of inorganic sediments and the separation of the aqueous phase from the hydrocarbon phase;

g5) separately extracting the hydrocarbon phase, the aqueous phase and the sediments from said tank.

Specifically, the aforesaid biosurfactants may advantageously be ramnolipid RLL and/or ramnolipid RRLL-based.

Rhamnolipid RLL is the compound (α-L-rhamnopyranosyl-α-L-rhamnopyranosyl-β-hydroxydecanoil-β-hydroxydecanoate), whereas rhamnolipid RRLL is the compound (2-O-α-L-rhamnopyranosyl-α-L-rhamnopyranosyl-β-hydroxydecanoil-β-hydroxydecanoate).

More in detail, the biosurfactants used are of microbial origin, and besides the rhamnolipids mentioned above, formed during the controlled fermentation process, they contain glycolipids, surface-active agents and biological emulsifiers. These biosurfactants are capable of emulsifying heavier hydrocarbon fractions in the bottom sludge, reducing the viscosity thereof and the tendency to precipitate.

Thanks to the action of these biosurfactants, during the settling step subsequent to the fluidisation by mixing the hydrocarbon phases remain in suspension, actually letting only the inorganic solids (sands, oxides) and the solid non-emulsifiable hydrocarbon phases (resins, lakes, asphaltenes, charcoal) precipitate. The settling further allows separating the hydrocarbon phase from the aqueous phase. Operatively, it is therefore possible to separately take the various phases directly from the tanks. The aqueous phase, substantially free from hydrocarbons, can be disposed of more easily, whereas the hydrocarbon phase, freed from solid sediments and from water, can be subject to the normal refining processes at lower costs.

According to a particularly advantageous optional feature of the present invention, the step g) of treating the bottom sludges is conducted using the same devices for generating pressurised jets to be used for the aforesaid subsequent introduction step a) of the aqueous mixture.

This makes it possible to conduct the operations relative to treating the bottom sludges and the operations relative to degassing in sequence, without the need for intermediate preparative operations involving opening the tank and inserting inside it specific treatment devices.

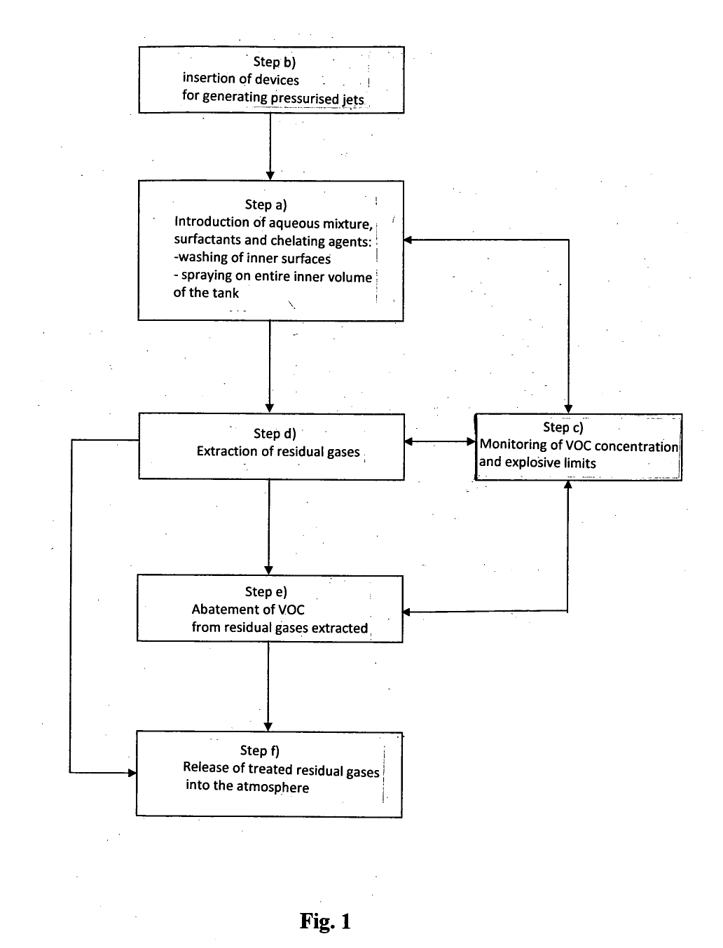

With reference to the flow diagram illustrated in FIG. 1, a particularly preferred form application of the present invention comprises in sequence the following steps:
(step b) inserting one or more devices for generating pressurised jets inside the tank by means of watertight chambers connected to the blind flanges of the manholes of the tank and communicating with the inside of the tank by means of holes made on the flanges using safe cutting techniques;
(step a) introducing inside the tank, kept closed, the aqueous mixture comprising surfactant substances or biosurfactants and chelating substances by means of the aforesaid devices, initially performing washing of the inner surfaces of the tank and continuing by spraying the mixture throughout the tank so as to reduce the concentration of the VOC to a predefined value functional to having an acceptable explosive limit;
(step c) regularly monitoring during step a) at least the concentration of the VOC and/or the explosive limits inside the tank to define the end of the aforesaid step a);
(step d) extracting the residual gases from the tank;
(step e) abating the VOC present in the residual gases extracted by means of at least one washing column using the aqueous solution to obtain concentrations of VOC compatible with the envisaged safety and environmental requisites;
(step c) regularly monitoring during step e) at least the concentration of the VOC and/or the explosive limits of the gaseous flow treated to assess the possibility of its release into the atmosphere;
(step f) release into the atmosphere of the residual gases subjected to abatement in step e).

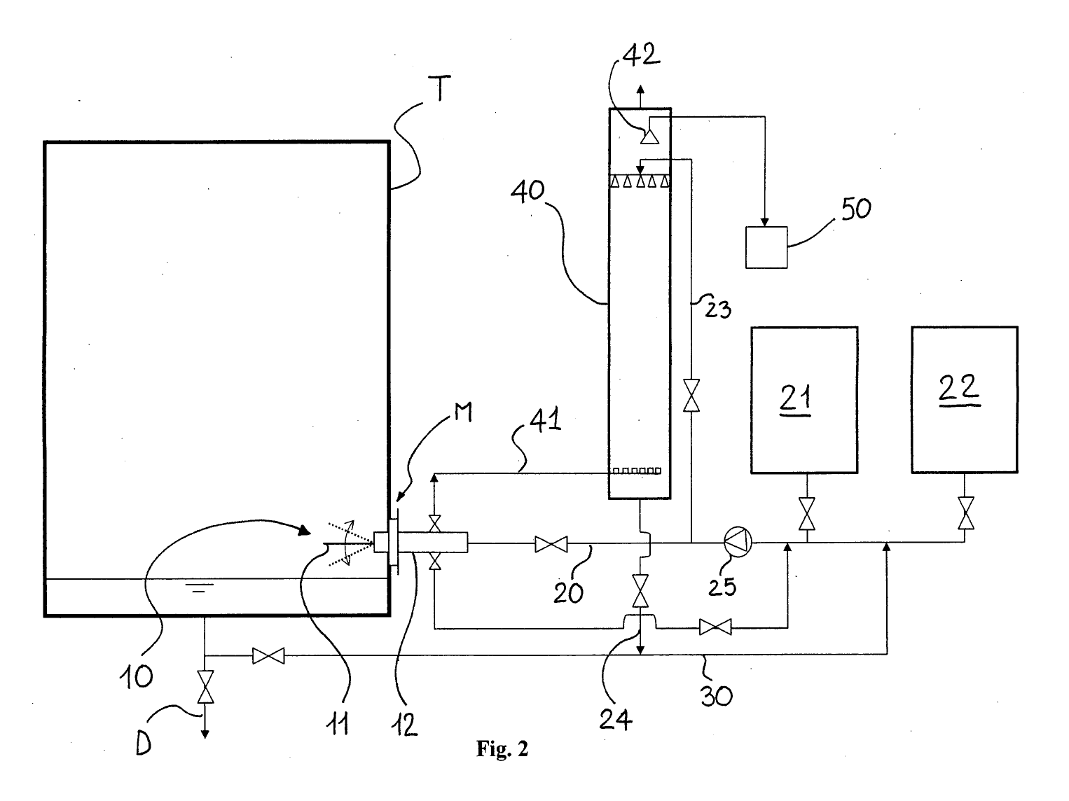

FIG. 2 shows a simplified diagram of a plant for implementing the method according to the invention according to a preferred application illustrated in FIG. 1.

The tank to be degassed is marked T and one of the manholes is marked M. The plant comprises at least one device 10 for generating pressurised jets, provided with a mobile gun 11. The device is inserted by means of a watertight chamber 12 connected to the blind flange of the manhole and communicating with the inside of the tank by means of a hole made on the flange, through which at least the gun 11 is inserted. The device 10 is connected by the valve means of the watertight chamber 12 to a supply line 20 of the aqueous solution. Such line 20, equipped with pumping means 25, is connected in parallel to a first tank 21 containing fresh aqueous mixture and to a second tank 22 collecting the aqueous mixture used. To such purpose the supply line 20 is connected to a recycling line 30 connected to the drainage ducts D of the bottom of the tank T. The plant comprises a washing column 40 for the abatement of the VOC present in the residual gases present in the tank at the end of the introduction step a) of the aqueous mixtue. The column 40 is supplied with aqueous mixture by means of a branch 23 of the supply line 20; the column is connected at the base by a drain line 24 connected to the recycling line 30. The residual gases of the tank enter at the base of the column 40 by means of an extraction line 41 communicating with the inside of the tank T by means of the valve means of the watertight chamber 12. The column 40 is provided at the top with means 42 for aspirating the gas from the tank T. Such aspiration means 42 are associated to a sampling probe, connected in turn to measurement means, indicated schematically by the numeral 50. Such measurement means 50 may comprise for example a multi-gas detector for H₂S, LEL (Lower Explosive Limit), O² and a photo-ionisation analyser for VOC.

The degassing method according to the invention brings many advantages, some of which are already described above.

The method makes it possible to significantly reduce the risks connected with degassing and opening operations of storage tanks without necessarily requiring inertisation of the tank. The VOC are in fact abated in the liquid phase directly inside the tank, kept closed, until acceptable explosive limits are achieved. At the moment of extracting the residual gases the concentration of VOC and relative explosive limit is in fact at an acceptable level as regards safety.

Moreover, the method according to the invention makes it possible to significantly reduce degassing times whilst operating in safety. From tests conducted in field the time needed to complete degassing using the method according to the invention is about ½ to ¼ of the time required using the traditional methods based on the use of suitable extractors and flushing with air

Below is a comparative example of degassing a tank of approx. 70 m in diameter with the roof positioned at about 2 m in height and containing a residue of crude oil bottom sludges.

Using a traditional method with extraction of the gas by means of pneumatic extractors with a capacity of 20,000 m³/h, degassing requires at least 4 days (96 hours), to reach an explosive limit of about 8%, a value which to be further reduced requires much longer times on account of the continual release of vapours by the residual bottom sludges.

Using the method according to the invention, two guns were introduced in diametrically opposite positions with a flow of 50 m³/h or less. An aqueous mixture of chelating substances and surfactants was used with a total percentage in weight of these agents of 0.006% of the total mixture. The mixture was sprayed at intervals of time over a 24-hour period, using one of the two guns alternately, amounting to a total aqueous mixture introduced of 50 m³ and a total spraying time of 1 hour. On a tank having the same geometric characteristics and in the same surrounding conditions as the case verified with the traditional method, the time required to abate the explosive level to about 3% took one day (24 hours).

Moreover, in the hours following degassing treatment no increase in the explosive level was observed, which does happen with the traditional treatments. This can be explained by the inhibiting effect on volatility of the VOC put into a liquid phase or already present in the phase itself (bottom sludges) deriving from the method according to the invention. In fact, the natural tendency of the residual product to release vapours can be countered.

The method according to the invention, thanks in particular to the use of the manholes, does not force operators to work in confined spaces with consequent simplification of the procedures and significant savings in time.

Moreover, the method according to the invention makes it possible to eliminate or significantly reduce the release of VOC, into the atmosphere. Again, while operating in safety in a confined area, it is in fact possible to further abate the concentrations of VOC in the residual gases extracted from the tank, should such values not be compatible from an environmental point of view with being released into the atmosphere.

As emphasised several times above, the method according to the invention makes it possible to operate with the tank constantly closed and therefore in a confined area with controlled emissions into the atmosphere.

The method according to the invention is operatively simple to implement, given that no complex procedures are envisaged nor is the use of sophisticated instrumentation required.

Moreover the method according to the invention can be conducted jointly with treatment of the bottom sludges of the tank.

The invention so conceived thereby achieves the predefined objectives.

Obviously its practical embodiments may assume forms and configurations different from those illustrated above while remaining within the scope of protection.

In addition, all the parts may be replaced by technically equivalent elements and any sizes, forms and materials may be used as needed.

1. Method for degassing a storage tank for liquid products containing volatile organic compounds or VOC, in particular petroleum products, comprising a step a) of introducing inside the tank, which is kept closed, an aqueous mixture comprising substances able to interact chemically and/or physically with the VOC in the gaseous state so as to encourage them both to pass from the gaseous phase to the liquid phase and to remain in the liquid phase, said introduction step being protracted until the concentration of the VOC is reduced to a predetermined value functional to having an acceptable explosive limit for the purposes of a possible opening of the tank in safety for internal inspection.

2. Method according to claim 1, wherein said aqueous mixture comprises one or more surfactant substances.

3. Method according to claim 2, wherein said one or more surfactant substances are chosen from the group comprising alkylphenol ethoxylate, ethoxylate alcohol, sodium metasilicate, sodium dioctylsulphosuccinate, polypropylene glycol, alkyl-diphenyl-ether disulphonate, polyoxyethylene stirilfenil ether sulphate, polyoxymethylene alkyl ethers, polyoxyethylene alkyl amines, polyoxyethylene sorbitan laurates, polyoxyethylene sorbitan stearates, polyoxyethylene sorbitan oleates, polyoxyethylene sorbitan trioleates, polyoxyethylene stirilfenil ethers, carboxy betaine, benzyl ammonium salts and/or imidazolin laurate.

4. Method according to one or more of the previous claims, wherein said aqueous mixture comprises one or more biosurfactants.

5. Method according to claim 4, wherein said one or more biosurfactants are chosen from the group comprising glycolipids, lipopeptides, lipopolysaccharides, phosopholipids, fatty acids/neutral lipids and/o proteins.

6. Method according to one or more of the claims from 2 to 5, wherein said surfactants and/or biosurfactants are present in said aqueous mixture with a percentage in weight of the total of 0.002% to 0.006%.

7. Method according to one or more of the previous claims, wherein said aqueous mixture comprises one or more chelating substances.

8. Method according to claim 6, wherein said one or more chelating substances are chosen from the group comprising EDTA and derivatives, acetyl acetate, polyamines, oxalates, carbonates, crown ethers, 2,2′-bipiridin, glycinate, nitrylotriacetate, dimercaptopropanol, penicillamine, deferoxamine mexylate, dimercaptosuccinic acid, trientine and/or Chlorella vulgaris green unicellular water microalgae.

9. Method according to claim 7 or 8, wherein said chelating substances are present in said aqueous mixture in a percentage in weight of the total of 0.001% to 0.004%.

10. Method according to one or more of the previous claims, wherein the introduction of said aqueous mixture inside said tank is performed by means of one or more devices for generating pressurised jets inside said tank.

11. Method according to claim 10, wherein said introduction step a) comprises spraying said aqueous mixture inside said tank by means of said pressurised jets, preferably throughout the whole volume of said tank.

12. Method according to claim 10 or 11, wherein said introduction step a) comprises washing the inner surfaces of said tank with said aqueous mixture by means of said pressurised jets, preferably said washing being performed at the beginning of said introduction step a).

13. Method according to claim 10, 11 or 12, comprising an step b) of inserting said devices inside said tank by means of watertight chambers.

14. Method according to claim 13, wherein said watertight chambers are connected to the blind flanges of the manholes of said tank and communicate with the inside of said tank by means of holes made on said flanges by cold cutting, the connection to the flanges of the manholes is made using attachment bolts, without any welding.

15. Method according to any of the previous claims, comprising a step c) of monitoring at least the concentration of the VOC and/or of the explosive limits of the gases present inside said tank.

16. Method according to claim 15, wherein said monitoring step c) is conducted on at least one flow of gas extracted from said tank.

17. Method according to claim 15 or 16, said monitoring step c) is conducted prior to, during and/or at the end of said introduction step a), preferably repeated at predefined intervals of time.

18. Method according to one or more of the previous claims, comprising an step d) of extractiing the residual gases present in the tank at the end of said introduction step a).

19. Method according to claim 10, 11 or 12 and claim 18, wherein said extraction of the residual gases present in the tank is conducted using the same devices for generating pressurised jets used for said introduction step a).

20. Method according to claim 18 or 19, comprising an step e) of abating the VOC present in the residual gases extracted from said tank.

21. Method according to claim 20, wherein the abatement of the VOC is conducted by combustion, condensation, absorption in the liquid phase and/or absorption with activated carbon.

22. Method according to claim 21, wherein the abatement in liquid phase envisages use of said aqueous mixture.

23. Method according to claim 15, 16 or 17 and claim 20, 21 or 22, said monitoring step c) is conducted prior to, during and/or at the end of said abatement step e) of the VOC in the residual gases extracted, preferably repeated at predefined intervals of time.

24. Method according to one or more of the claims from 17 to 23, comprising a step f) of releasing the residual gases extracted from said tank in the atmosphere, the release occurring preferably in function of the concentration values measured in said monitoring step c).

25. Method according to claims 20 and 24, wherein said release step f) occurs subsequently to said abatement step e) of the VOC.

26. Method according to one or more of the previous claims, wherein said introduction step a) is preceded by a step g) of treating of the bottom sludges present in said tank.

27. Method according to claim 26, wherein said treatment step g) of the bottom sludges comprises the sub-steps of:
g1) breaking up the bottom sludge the bottom sludges by means of one or more pressurised jets;
g2) fluidising the bottom sludges by mixing by the action of said jets;
g3) dispersing biosurfactants able of emulsifying heavier hydrocarbon fractions, reducing the viscosity thereof and the tendency to precipitate;
g4) settling the contents of said tank interrupting the mixing thereof, so as to allow the precipitation of inorganic sediments and the separation of the aqueous phase from the hydrocarbon phase;
g5) separately extracting the hydrocarbon phase, the aqueous phase and the sediments from said tank.

28. Method according to claim 27, wherein said biosurfactants are ramnolipid RLL and/or ramnolipid RRLL-based.

29. Method according to claim 10, 11 or 12 and claim 27 or 28, wherein said treatment step g) of the bottom sludges is conducted using the same devices for generating pressurised jets to be used for said introduction step a).

30. Plant for implementing the degassing method according to one or more of the previous claims.
